(19) United States
(12) Patent Application Publication
(10) Pub. No.: US 2016/0136109 A1
(43) Pub. Date: May 19, 2016

(54) COMPOSITIONS FOR TISSUE STABILIZATION

(71) Applicant: Vatrix Medical, Inc., Maple Grove, MN (US)

(72) Inventors: Jason C. Isenburg, Victoria, MN (US); Matthew F. Ogle, Edina, MN (US)

(21) Appl. No.: 15/005,821

(22) Filed: Jan. 25, 2016

Related U.S. Application Data
(62) Division of application No. 12/617,322, filed on Nov. 12, 2009.
(60) Provisional application No. 61/113,881, filed on Nov. 12, 2008.

(51) Int. Cl. A61K 31/11

(52) U.S. Cl. CPC A61K 31/11

(57) ABSTRACT

Collagen crosslinking/stabilization composition optionally in combination with elastin crosslinking composition as treatment of vascular aneurysms, methods of using the compositions, especially with respect to in vivo procedures are described. The treatment is achieved through the delivery of an effective amount of crosslinking/stabilization composition to the site of the aneurysm. The crosslinking/stabilization agent may be embedded in a delivery composition and delivered to the site of aneurysm using a delivery device. The site of the aneurysm may be isolated for treatment using the delivery device. The elastin stabilization agent may be simultaneously or sequentially delivered with the collagen crosslinking/stabilization agent for the treatment of vascular aneurysms in the isolated section of blood vessel.

### CROSS REFERENCE TO RELATED APPLICATIONS

This application is a divisional of copending U.S. patent application Ser. No. 12/617,322 filed on Nov. 12, 2009 to Isenburg et al., entitled “Compositions for Tissue Stabilization,” which claims priority to U.S. provisional patent application Ser. No. 61/113,881, filed on Nov. 12, 2008 to Isenburg et al., entitled “Compositions for Tissue Stabilization,” both of which are incorporated herein by reference.

### FIELD OF THE INVENTION

The inventions, in general, are related to a connective tissue stabilization composition to treat vascular aneurysm. The inventions are further related to methods of using the compositions, especially with respect to in vivo procedures.

### BACKGROUND

Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the popliteal and femoral arteries are prevalent locations of this vascular pathology. The occurrence of such peripheral aneurysms appears to be strongly associated with the presence of aneurysms in other locations, as it has been estimated that 30 to 60% of peripheral aneurysm patients also have an AAA.

Aneurysms grow over a period of years and pose great risks to health. Aneurysms have the potential to dissect or rupture, causing massive bleeding, stroke, and hemorrhagic shock, which can be fatal in more than 80% of cases. AAAs are a serious health concern, specifically for the aging population, being among the top ten causes of death for patients older than 50. The estimated incidence for abdominal aortic aneurysm is about 50 in every 100,000 persons per year. Approximately 60,000 operations are performed each year in the U.S. for AAAs alone. In children, AAAs can result from blunt abdominal injury or from Marfan's syndrome, a defect in elastic fiber formation in walls of major arteries, such as the aorta.

Aneurysms can be caused by any of a large class of degenerative diseases and pathologies including atherosclerotic disease, defects in arterial components, genetic susceptibilities, and high blood pressure, among others, and can develop silently over a period of years. The hallmarks of aneurysms include enzymatic degradation of vascular structural proteins such as elastin, inflammatory infiltrates, calcification, and eventual overall destruction of the vascular architecture.

Current methods of treatment for diagnosed aneurysms are generally limited to invasive surgical techniques. After initial diagnosis of a small aneurysm, the most common medical approach is to follow up the development of the aneurysm and after reaching a pre-determined size (e.g., about 5 cm in diameter), surgical treatment is applied. Current surgical treatments generally are limited to either an endovascular stent graft repair or optionally complete replacement of the diseased vessel with a vascular graft. While such surgical treatments can save lives and improve quality of life for those suffering aneurysm, dangers beyond those of the surgery itself still exist for the patient due to possible post-surgery complications (e.g., neurological injuries, bleeding, or stroke) as well as device-related complications (e.g., thrombosis, leakage, or failure).

Depending upon the location or anatomy of the aneurysm, the danger of an invasive surgical procedure may outweigh the possible benefits of the procedure, for instance in the case of an aneurysm deep in the brain, leaving the sufferer with very little in the way of treatment options. Moreover, surgical treatments may not always provide a permanent solution, as vascular grafts can loosen and dislodge should the aneurysm progress following the corrective surgery. Generally, most of the current treatment options for aneurysm are mechanical bridges. For some patients, the particular nature of the aneurysm or the condition of the patient makes the patient unsuitable for graft repair.

Aneurysm is not the only condition for which enzymatic degradation of structural proteins is a hallmark. Other conditions in which structural protein degradation appears to play a key role include Marfan's syndrome, supravalvular aortic stenosis, and chronic obstructive pulmonary disease (COPD). For those afflicted, such conditions lead to, at the very least, a lowered quality of life and often, premature death.

### SUMMARY OF THE INVENTION

In a first aspect, the invention pertains to a therapeutic composition that comprises an elastin stabilization agent, a collagen stabilization agent and a biocompatible delivery vehicle.

The collagen stabilization agent comprises a compound that is a polyamine, a photo-catalytic dye, genipin, an epoxide, an azide ester, or a combination thereof. The delivery vehicle can comprise a sustained release drug delivery vehicle, a hydrogel, a nano encapsulating particle material, a perivascular drug delivery vehicle, an endovascular drug delivery vehicle, a stent or a combination thereof. In some embodiments, the therapeutic composition comprises the compound in a concentration of from about 0.0001% to about 10% (w/v). In one embodiment, the collagen stabilization agent comprises a diamine and a carbodiimide. In another embodiment, the carbodiimide is 1-ethyl-3-(3-dimethyl aminopropyl)-carbodiimide, N,N′-dicyclohexylcarbodiimide, N,N′-diisopropyl carbodiimide or combinations thereof. In one embodiment, the compound is a photo catalytic dye, which is methylene blue, methylene green, rose bengal, riboflavin, proflavin, fluorescein, eosin, pyridoxal-5-phosphate, or combinations thereof. In another embodiment, the compound comprises triglycidylamine. In yet another embodiment, the compound comprises diphenylphosphoryl azide.

In some embodiments, the elastin stabilization agent comprises a compound that is tannic acid or a derivative thereof, a flavonoid or a flavonoid derivative, a flavolignan or a flavolignan derivative, a phenolic rhizome or a phenolic rhizome derivative, a flavan-3-ol or a flavan-3-ol derivative, an ellagic acid or an ellagic acid derivative, a procyanidin or a procyanidin derivative, anthocyanins, quercetin, (+)-catechin, (−)-epicatechin, pentagalloylglucose, nobotanin, epigallocatechin gallate, gallotannins, an extract of olive oil or a derivative of an extract of olive oil, cocoa bean or a derivative of a cocoa bean, Camellia or a derivative of camellia, licorice or a derivative of licorice, sea whip or a derivative of sea whip, aloe vera or a derivative of aloe vera, chamomile or a derivative of chamomile, a combination thereof, or a pharmaceutically acceptable salt thereof. In one embodiment, the compound is pentagalloylglucose.

In a second aspect, the invention pertains to a method for the stabilization of a blood vessel in a living subject. The method comprises applying an effective amount of a collagen stabilization agent and an elastin stabilization agent to an isolated section within the interior of the blood vessel. The blood vessel comprises collagen, which has a plurality of reactive groups and elastin. The collagen stabilization agent comprises a delivery vehicle and a compound that reacts with reactive groups of the collagen. The elastin stabilization agent comprises a delivery vehicle and a compound that crosslinks elastin. In some embodiments, the method further comprises aspirating the isolated section of the blood vessel prior to the application of the stabilization agent. In one embodiment, the stabilization agent is applied for about 1 min to about 4 hours. In another embodiment, the stabilization agent is applied for about 5 min. to about 2 hours. In one embodiment, the compound in the collagen stabilization agent is an aldehyde with at least two aldehyde groups, a polyamine with carbodiimide, a photo-catalytic dye, genipin, an epoxide, an azide ester, or a combination thereof. In one embodiment, the delivery vehicle comprises nano encapsulating particles.

In some embodiments, the method further comprises aspirating the vessel to remove the stabilization agent and applying a second quantity of the stabilization agent after performing the aspiration. In one embodiment, the stabilization agent applied in the second application has a different composition from the collagen stabilization agent of the first application. In some embodiments, the collagen stabilization agent is applied simultaneously with the elastin stabilization agent, which comprises a phenolic compound with a hydrophobic core. In some other embodiments, the collagen stabilization agent is applied sequentially with the elastin stabilization agent, which comprises a phenolic compound with a hydrophobic core. In some embodiments, the method further comprises an aspiration, a rinse, or combinations of the aspiration and rinse steps. In one embodiment, the rinse comprises an amino acid, serum albumin or a combination thereof. In one embodiment, the isolated section of the blood vessel comprises aneurysm.

In a third aspect, the invention pertains to a method for in vivo stabilization of a blood vessel, comprising rinsing an isolated section of the blood vessel with a toxicity reducing composition following the treatment of the isolated section of blood vessel with a multifunctional aldehyde. The toxicity reducing composition comprises a toxicity reducing agent. In one embodiment, the multifunctional aldehyde is glutaraldehyde. In some embodiments, the toxicity reducing agent comprises a nonionic surfactant. In some embodiments, the toxicity reducing composition comprises amines. In one embodiment, the amine comprises an amino acid. In some embodiments, the toxicity reducing composition comprises inorganic sulfur-oxygen containing anions, organic sulfates, ammonium salts, surfactants, or a combination thereof. In one embodiment, the sulfur-oxygen anion comprises sulfate anions SO₄⁻², thiosulfate anions S₂O₄⁻², bisulfate HSO₄⁻, or a combination thereof. In another embodiment, the organic sulfates comprises methyl sulfate CH₃O₄S⁻, dimethyl sulfate (CH₃)₂O₄S and dodecyl sulfate CH₃(CH₂)₁₁O₄S⁻ as well as protonated forms thereof. In one embodiment, the ammonium salt comprises ammonium chloride, ammonium hydroxide or other suitable salt thereof. In another embodiment, the surfactants comprises aliphatic fatty acid esters, polypropyleneglycol fatty acid esters, glycerol fatty acid esters, polyalkylene ethers, polyoxyethylene oleyl ether, polyoxyethylene cetyl ether, polyethylene glycol p-isooctyl phenyl ethers, polyoxyethylene sorbitan esters, or a combination thereof. In general, the toxicity reducing agents have a concentration from about 0.005 molar (M) to about 3 M,

In a fourth aspect, the invention pertains to a method for the stabilization of a blood vessel in a living subject, comprising applying an effective amount of a collagen stabilization agent to an isolated section within the interior of the blood vessel. The collagen stabilization agent comprises a compound that is a polyamine, a photo-catalytic dye, genipin, an epoxide, an azide ester, or a combination thereof.

### BRIEF DESCRIPTION OF THE DRAWINGS

FIG. 1 is a schematic diagram of a section of blood vessel containing aneurysm being isolated by a sealing device, which has a by-pass channel to allow blood flow.

FIG. 2 is a schematic diagram of a section of aorta below renal arteries containing aneurysm being isolated by introducer sheaths and a sealing catheter for treatment.

FIG. 3A is a photograph showing a perspective view of a porcine aorta cut transversely into ring segments and a photograph of the top view of the ring segment being cut open.

FIG. 3B is a set of photographs of the treated ring segments that were cut open and allowed to relax following various treatments of the tissue.

FIG. 3C is a plot showing the opening angle of aortic rings compared for different treatments.

FIG. 4 is a plot showing the percentage digestion of treated tissues compared for different treatments of the tissue following exposure to collagenase.

FIG. 5 is a plot showing the digestion of portions of porcine carotid arteries treated with different therapeutic compositions having varied abilities to resist enzymatic degradation to elastase.

FIG. 6 is a plot showing stress versus strain for portions of porcine carotid arteries treated with different therapeutic compositions having varied uniaxial tensile strength.

FIG. 7 is a plot showing stress versus percent elongation of porcine aortic rings treated with different therapeutic compositions, showing varied tensile strength.

FIG. 8 is a bar graph comparison of the opening angles of porcine aortic rings from different treatments outlined in Example 6.

### DETAILED DESCRIPTION

Collagen crosslinking/stabilization agent can provide a high degree of stabilization of vascular tissue associated with aneurysms and other degeneration of blood vessels. In some embodiments, the collagen crosslinking/stabilization agent can be combined with an elastin stabilizing agent. The treatment agents can be contacted with the tissue simultaneously or sequentially. Multi-functional reagents, such as glutaraldehyde, genipin, acyl azide, and epoxyamine, are known to cross-link functional groups in collagen thereby stabilizing collagen and tissue having a collagen component. These collagen crosslinking/stabilization agents can be incorporated into a therapeutic composition to stabilize tissues associated with an aneurysm or the like. The therapeutic composition can further comprise an elastin stabilizing agent, such as pentagalloylglucose (PGG). The combination of a collagen crosslinking/stabilization agent and an elastin stabilization agent can be particularly effective with respect to stabilizing vascular tissue, such as an aneurysm tissue since collagen and elastin are important components of the extracellular matrix of connective tissue, such as blood vessels.

In general, connective tissue targeted with the therapeutic agent(s) or composition(s) can be stabilized so as to be less susceptible to protein degradation as well as having improved mechanical strength to resist distortion of the natural shape and possible bursting. Some collagen crosslinking agents can have significant cytotoxicity, and techniques are described herein for the delivery into isolated portions of a blood vessel to provide for removal of the agent at the end of the procedure. Photoactivated crosslinking composition can be effective since the unactivated compound can have less toxicity. A separate rinsing step can be applied after the treatment to deactivate the residual reactive agent at the site of treatment.

In some embodiments, the collagen crosslinking/stabilization agents can be administered alone. In other embodiments, the collagen crosslinking/stabilization agents can be combined with elastin stabilization agent. In yet other embodiments, the collagen crosslinking/stabilization agent and elastin stabilization agent can be administered in separate application steps sequentially to the site of aneurysm. The collagen crosslinking/stabilization agent and elastin stabilization agent can each have an appropriate application time, composition, and concentration. The therapeutic compositions maybe formulated with delivery vehicles and delivered to the site of treatment with a delivery device. The treatment parameters such as concentration, composition, delivery vehicle, application device and method of delivery can be adjusted to suit variety of application in stabilizing tissues with collagen and/or elastin component.

Connective tissue generally comprises individual cells not directly attached to one another and held within an extracellular matrix. The extracellular matrix, in turn, comprises compositions excreted by specific cells with specific mechanical properties, which include, for example, fibrous components such as collagen fibers and elastin fibers. Connective tissue can assume widely divergent architectures. Blood vessels generally involve connective tissue, for example, with a thin layer of endothelial cells lining the blood vessel. The degradation of the tissue can be brought about due to any of a variety of mechanisms and/or conditions including, for example, those associated with aneurysm, atherosclerotic disease, genetic susceptibilities, blunt force injury, Marfan's syndrome, and the like.

Recent techniques have been developed to track the progress of the aneurysm using a laboratory test, such as a blood test, a urine test or a combination thereof. This is described further in copending published U.S. Patent Application No. 2009/0186370 to Ogle et al., entitled “Diagnostic Biomarkers for Vascular Aneurysm,” incorporated herein by reference. Objectives of this testing includes, for example, earlier detection of aneurysms and an alternative approach for the tracking of the development of the disease. Once the aneurysm is identified and has progressed to a stage of initiating treatment, imaging generally is used to identify the location of the aneurysm and to assess the severity of the problem and to identify an approach for the treatment procedure.

Methods for diagnosing and identifying the degree of aneurysm expansion are available due to developments in high resolution imaging technology (CT, MRI). Various appropriate contrast agents can be used to enhance the imaging. The use of magnetic resonance and CT imaging techniques to guide procedures on aneurysms is described further in U.S. Pat. No. 6,463,317 to Kucharczyk et al., entitled “Device and Method for the Endovascular Treatment of Aneurysms,” and U.S. Pat. No. 6,793,664 to Mazzocchi et al., entitled “System and Method of Minimally-Invasive Exovascular Aneurysm Treatment,” both of which are incorporated herein by reference.

At an aneurysm, blood vessels exhibit degradation of the tissue. Due to the blood pressure in the vessel, as the tissue of blood vessel weakens, the vessel generally expands at the location of weakness. The expansion further effects flow in the vicinity of the expansion. Upon further weakening of the vessel, the vessel can rupture due to the pressure in the vessel with corresponding deleterious effects. In some embodiments described herein, the blood vessel can be sculptured to more closely resemble the natural shape of the vessel along with stabilizing the tissue such that more normal function of the vessel can be expected.

Specifically, at an aneurysm, collagen is present throughout the aneurysm tissue. See, for example, Loftus I M, Thompson M M. Vasc Med 2002; 7(2): 117-133, incorporated herein by reference. In the course of aneurysm development, it has been suggested that the processes of degradation and regeneration of collagen alternates. Once the collagen degradation reaches a particular degree, the rupture of the aneurysm tissue may occur. See, for example, Choke E, Cockerill G, Wilson W R, et al. Eur J Vasc Endovasc Surg 2005; 30(3): 227-244, incorporated herein by reference. Stabilization of collagen in aneurysm tissue can be an effective aspect for treating vessel damage associated with an aneurysm.

Evidence also suggests that elastin degradation contributes significantly to the aneurysm in blood vessels. Elastin stabilization compositions comprising phenolic type compounds have been described in detail in U.S. Pat. No. 7,252,834 (the '834 patent) and copending continuation U.S. patent application Ser. No. 11/827,121 the '543 patent, now issued U.S. Pat. No. 7,713,543 (the '543 patent), to Vyavahare et al., entitled “Elastin Stabilization of Connective Tissue”, both are incorporated herein by reference. The elastin stabilization agents can reduce or eliminate further enzymatic degradation of the tissue and mechanically stabilize the elastin proteins fibers.

In general, protein crosslinking agents bond with functional groups of proteins, which are generally widely found in proteins. While protein crosslinking agents generally are broadly reactive, some agents have some particular specificity for more extensive effective crosslinking for certain proteins. In particular, structural proteins, such as elastin and collagen, have amino acid sequences that can use high percentages of certain amino acids that can provide for targeting crosslinked structures with more stability for certain crosslinking compositions. Thus, we refer herein to elastin stabilization agents and collagen crosslinking agents due to preferential bonding to these proteins, which the agents may have some crosslinking with a broad range of proteins.

Since elastin and collagen comprise two very significant structural components of connective tissue, the stabilization and/or crosslinking of both of these fibrous proteins can be particularly effective at stabilizing an aneurysm. The stabilization of elastin can be particularly effective using the stabilization compositions described herein that generally protect hydrophobic domains of the elastic protein through steric effects to inhibit degradation of the elastin by elastase. The crosslinking of collagen is well established for the in vitro treatment of tissue to form a prosthetic material. The complete crosslinking of the tissue essentially kills all viable cells and removes any antigenic response such that xenograft tissue can be used, e.g., porcine tissue for human use. Such extensive crosslinking is not suitable for in vivo use for many reasons. However, a lighter crosslinking can provide some mechanical stabilization without inducing extensive cytotoxicity. The in vivo use has fundamental differences from the in vitro use of crosslinking agents. Specific crosslinking agents have been identified that are particularly effective at crosslinking collagen based on the specific fibrous structure and amino acid composition.

The use of an elastin stabilization agent in combination with glutaraldehyde is described generally in the '834 patent and the '543 patent. These references do not describe the use of collagen crosslinking agents other than glutaraldehyde, which may be more desirable in some applications for in vivo use. Also, this patent does not describe particular application approaches to isolated regions of a blood vessel to provide a greater control on the delivery process and for the removal of unreacted crosslinking agent at the end of treatment.

The delivery of glutaraldehyde or carbodiimide into an isolated section of a blood vessel is described in published U.S. patent application 2001/0029349A to Leschinsky, entitled “Method and Apparatus for Treating Aneurysm,” incorporated herein by reference. This reference does not describe any specific information regarding the treatment solutions. Furthermore, this reference does not describe any specific treatment process other than to imply that the treatment can be made with “no time constraints” due to the profusion past the device.

The treatment agents can be delivered or administered acutely or chronically according to various delivery methods, such as sustained release methods and/or with suitable delivery agents, such as microsphere carriers, encapsulating nanoparticles, hydrogels, and/or a combination thereof, for example delivery system such as those described in the published U.S. Patent Application No. 2009/021465412/390,156 to Isenburg et al. (the '654 application) entitled “Treatment of Aneurysm with Application of Connective Tissue Stabilization Agent in Combination with a Delivery System”, incorporated herein by reference.

The therapeutic composition disclosed herein can be directed to localized delivery to stabilize the collagen and/or elastin component of connective tissue, and in particular, blood vessels or other vessels. It should be understood that while a therapeutic composition can be directed in some embodiments to the stabilization of blood vessels susceptible to the formation of aneurysms, in other embodiments, other organs, other diseases and/or other conditions can be treated. In particular, the disclosed treatment agents and treatment protocols may be applicable to any animal or human connective tissue that comprises a collagen component and/or elastin component of an extracellular matrix. It should be also understood that while the therapeutic compositions are named as elastin stabilization agents or collagen stabilization agents, in actual tissue application, the agents may interact with additional proteins and not necessarily only elastin or collagen at the site of treatment to stabilize the tissue. However, the therapeutic agents generally are believed to be particularly effective with respect to the specified therapeutic roles.

As described herein, less invasive procedures can be used to deliver chemical stabilizing agents to stabilize the tissue in the vicinity of the aneurysm. Some level of structural remolding can be performed in conjunction with the chemical stabilization. In contrast, surgical treatment of aneurysms can involve endovascular stent graft repair (placement of a tube inside the vessel) or complete replacement of the diseased aorta or other blood vessel with an artificial vascular graft. Surgical treatment of aneurysms saves thousands of lives every year and improves quality of life. However, survival rates can drop to only 50% at 10 years post-operative due to surgery-related complications or device-related problems. In addition, endovascular stents are anatomically appropriate for only 30% to 60% of AAA patients at the outset and present the risk of endoleaks and graft displacement. Moreover, open surgery for full-size graft insertion is highly invasive, limiting its use to those patients with the ability to tolerate high operative risk. Early interventions for these potentially debilitating and life-threatening vascular pathologies may be advantageous since age is one of the major risk factors associated with the current approaches to treat aneurysms.

Devices and methods for effective delivery of therapeutic composition disclosed herein are described in published U.S. Patent Application No. 2010/0016833 to Isenburg et al., entitled “Devices for the Treatment of Vascular Aneurysm,” (hereinafter the '833 application) incorporated herein by reference. The isolation of the blood vessel wall can be performed with device designs that provide for continued blood flow past the isolated region based on a specific conduit or an appropriately designed opening through the structure providing the isolated treatment location. The device generally comprises a shaft and a sealing element. The devices can be introduced into the vessel using techniques for the delivery of catheters and the like using less invasive procedures. The sealing portion of the device can be oriented within the vessel to be positioned in the vicinity of the aneurysm so that the resulting enclosed volume encompasses at least a portion of the aneurysm.

Additionally, devices with sealing balloons that can adjustably isolate a section of a vessel to create an adjustable isolated volume are described in U.S. Pat. No. 8,444,624 to Ogle et al, (the '624 patent) entitled “Vascular Medical Devices with Sealing Elements and Procedures for the Treatment of Isolated Vessel Sections,” incorporated herein by reference. A sealing balloon associated with an introducer sheath can be used to seal a portion of a blood vessel through the femoral artery in a patient's leg. The location of the balloons can be adjusted for example to isolate an entire blood vessel region that has aneurysm or showing signs of aneurysm, especially a section of the aorta below the renal arteries.

In some embodiments, blood can be withdrawn from the isolated or enclosed volume.

Withdrawal of the blood from the isolated volume reduces the pressure below the blood pressure within the vessel. If the vessel has appropriate elasticity, the aneurysm can respond to the reduced pressure by reducing the volume of the aneurysm with at least partial return of the vessel to its natural shape. In some embodiments, a pretreatment step is performed before the delivery of the therapeutic composition. The pretreatment step comprises the delivery of a thrombolytic composition to break up thrombus in the isolated blood vessel.

After the isolated section of the blood vessel is reasonably cleaned up, a therapeutic agent can be introduced. The therapeutic agent can stabilize the tissue from further degradation and mechanical instability. The tissue stabilization can reduce the chance of rupture of the aneurysm with corresponding serious or potentially fatal consequences. Through the use of the combination of withdrawal of blood and the addition of a stabilization agent, the aneurysm can be stabilized in a less distorted shape. The blood withdrawal and addition/aspiration of stabilization agent(s) can be performed multiple times with same or different agents until desired effect is reached. For example, after the deployment of the device and subsequent blood withdrawal, an effective amount of a therapeutic agent, such as a collagen crosslinking/stabilization agent and/or an elastin stabilization agent, can be administered for a period of time to stabilize the tissue. The collagen crosslinking/stabilization agent is then aspirated from the blood vessel. A detoxification agent such as amino acid or serum albumin may then be administered to remove any residual harmful agent.

In general, mass to volume percentage (% w/v) is used to define the concentration of the reagents used herein. Mass to volume percentage is the relationship of a solute to a solvent expressed as grams of solute per milliliter of the total solution, as used herein and as standard in the particular art. For example, 0.06 g of pentagalloylglucose (PGG) in 100 mL of solution is considered a 0.06% w/v PGG solution. In some embodiments, the concentration of the effective component of the therapeutic composition can be in the range of about 0.0001% to about 10% (w/v). In other embodiments, the concentration can be in the range of about 0.01% to about 1%. The isolated section of the vessel can be treated with the therapeutic composition for about 1 min to about 4 hours. In some embodiments, the treatment can last about 5 min to about 1 hour.

The therapeutic composition and method of using the composition described herein can provide treatments to inhibit and/or reverse the progression of aneurysm, prevent further weakening and dilation of the vessel wall. Delivery of the therapeutic composition can be carried out in a less invasive format that reduces the recovery time and risk of the procedure to the patient. The use of devices that isolate portions of the blood vessel provide for safer delivery application and/or removal of remaining therapeutic agents as well as significant control of the design and implementation of a treatment protocol that can involve a plurality of steps with treatment agents or other procedures as described herein.

### Collagen Stabilization Agents

Collagen stabilization agents are generally multi-functional agents that cross-link reactive groups, such as amino, thiol, hydroxyl, or carbonyl groups, in collagen and/or nearby proteins. By binding to and crosslinking collagen, the multi-functional agents can increase the mechanical strength of the tissue. In the case of aneurysm, the increased mechanical strength of aneurysm vessel increases the tolerance of the treated aneurysm tissue to burst pressure, thus decrease the risk of rupture of the vessel. Some collagen stabilization agent maybe used for facile in vivo treatment employing a delivery device. Agents may have acute in vivo toxicity such that isolation of the treatment site during the treatment process can be very advantageous. Some collagen stabilization agents maybe used for slow release at the site of the aneurysm, for example, in the form of coating of a stent or embedded in surgical girdle that wraps around the aneurysm vessel.

Tissue treated with collagen crosslinking/stabilization agent with or without combination with elastin stabilization agent may exhibit enhanced mechanical property, resistance to enzymatic degradation such as elastase and collagenase, and a higher thermal denaturation temperature. The thermal denaturation temperatures (T_{d}) are common indicators of collagen crosslinking density, which can be measured using a differential scanning calorimeter (DSC). Detailed experimental results are discussed in Examples 1-6 for tissue treated in vitro as a model for in vivo delivery.

Glutaraldehyde and Other Bifunctional Aldehydes

Glutaraldehyde has been used as an in vitro tissue fixative for bio-prosthetic heart valves and other bioprosthetic tissue due to its ability to crosslink collagen, sterilize tissue, and reduce tissue antigenicity. Other bi-functional or multi-functional aldehyde compounds can be similarly used. Glutaraldehyde and other multi-functional aldehyde compounds bind to and stabilize collagen in the wall of the vessel. In particular, glutaraldehyde self-polymerizes to form polymer chains that are believed to be effective at crosslinking between collagen fibers.

Glutaraldehyde polymerizes with itself and/or with nearby active groups from collagen and/or other proteins creating crosslinks in the treated tissue. The chemical crosslinks in the tissue can contribute to increased resistance to degradation of the treated tissue. However, residual unreacted free aldehyde groups from glutaraldehyde can contribute with regards to toxicity and calcification. Treatment of bioprosthetic tissue to reduce toxicity is described in U.S. Pat. No. 6,471,723 to Ashworth et al., entitled “Biocompatible Prosthetic Tissue,” incorporated herein by reference. The use of these agents is described further below in the context of rinsing the blood vessels in vivo to reduce cytotoxicity.

By binding to and crosslinking collagen, glutaraldehyde increases the mechanical strength of the tissue. The in vivo application of the glutaraldehyde alone and in combination with PGG have been briefly discussed in the '834 patent with respect to treatment of aneurysms. For in vivo treatment directly at the site of the aneurysm inside a blood vessel, however, the amount of glutaraldehyde, treatment concentration, treatment time, toxicity control agent etc. used can be selected to achieve desired treatment effects while avoiding undesirable effects from excessive treatment, such as excessive cellular toxicity and over-stiffening of the vessel well. Preliminary experiment results using glutaraldehyde and/or an elastin stabilizer such as PGG or tannic acid have been presented and discussed in further detail in Examples 1-6.

Diamines/Carbodiimides

Alternatively or additionally, collagen stabilizing agents can comprise diamines, triamines or other polyamines, generally with at least two free primary amine groups, such as 1,6-hexanediamine and 1,7-heptanediamine. The diamines bond to carboxyl groups in proteins to form a crosslinked structure. It has been found that coupling agents and coupling enhancers facilitate this crosslinking/stabilization process with diamines. For example, suitable coupling agents include carbodiimides, such as 1-ethyl-3-(3-dimethyl aminopropyl)-carbodiimide (EDC) and/or n-hydroxysuccinimide (NHS). The carbodiimides function as a coupling agent in the crosslinking/stabilization reaction, and are generally used along with a coupling enhancer. For example, EDC can be used in conjunction with N-hydroxysulfosuccinimide (Sulfo-NHS), which acts as an enhancer to the reaction. Other suitable coupling enhancers include, for example, hydroxybenzotriazole (HOBt), Dimethylaminepyridine (DMAP), and NHS. By coupling the amine and carboxyl groups within the tissue, a treatment with diamines creates amide bonds that form chemical crosslinks between and/or within proteins, thus stabilizing the tissue. In vitro crosslinking/stabilization of bioprosthetic tissue with polyamines along with coupling agents and/or coupling enhancers is described further in published U.S. patent application 2006/0159641A to Girardot et al., entitled “Variably Crosslinked Tissue,” incorporated herein by reference.

Photo-Oxidation

Collagen stabilization in tissue can also be triggered by a light sensitive dye, similar to the PhotoFix™ technology used by Carbomedics for bioprosthetic heart valves. Photo-oxidation fixation relies on the use of a photoactive dye as a catalyst. Suitable photo activated dyes include, for example, methylene blue, methylene green, rose bengal, riboflavin, proflavin, fluorescein, eosin, pyridoxal-5-phosphate and combinations thereof. The dye catalyst aids in the conversion of amino acids within the tissue, subsequently allowing for crosslink formation between the converted amino acid and nearby amino acids to stabilize the tissue. See, for example, the following articles: Adams A K, Talman E A, Campbell L, et al. J Biomed Mater Res 2001; 57(4): 582-587 and Meuris B, Phillips R, Moore M A, et al. Artif Organs 2003; 27(6): 537-543), both of which are incorporated by reference.

Methods for ex vivo cross-linking collagen with a photo catalyst are described further in U.S. Pat. No. 5,147,514 to Mechanic et al, entitled “Process for cross-linking collage nous material and resulting product,” incorporated herein by reference. Photo-oxidation requires exposure of the treated tissue to a light source. In the case of aneurysm treatment, such light source can be supplied by for example, optical fiber. Additionally, oxygen may be supplied to the therapeutic composition to facilitate the completion of the photo-oxidative reaction. For example, oxygen can be introduced to the composition by mixing strongly in an oxygen atmosphere, which can be enriched in oxygen relative to air, if desired]

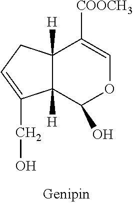 [SMILES: COC(=O)C1=CO[C@@H](O)[C@@H]2C(CO)=CC[C@H]12]

Genipin is a naturally occurring plant compound capable of crosslinking with collagen to stabilize connective tissue. Stereoisomers and mixtures thereof, such as racemic synthetic genipin, can be similarly used. Similarly, functionally active derivatives thereof can also be used to crosslink collagen. A person of ordinary skill in the art can make minor changes to the genipin structure and test the efficacy of the crosslinking based on the teachings herein to evaluate their remaining activity. Genipin is known to react with amino acids and/or proteins to form a dark blue pigment, which has been historically used as a food dye.

It has been shown that genipin is a relatively non-toxic compound for collagen crosslinking/stabilization. See the description in the following articles: Sung H W, Chang Y, Chiu C T, et al. Biomaterials 1999; 20: 1759-1772 and Sung H W, Chang W H, Ma C Y, et al. J Biomed Mater Res 2003; 64A: 427-438, both of which are incorporated by reference. Methods for ex vivo cross-linking collagen for bioprosthetic tissue are described in U.S. Pat. No. 6,608,040 to Sung et al., entitled “Chemical Modification of Biomedical Materials with Genipin,” incorporated herein by reference. Additionally, genipin has been used in vascular stent as a collagen crosslinking/stabilization agent for treating vulnerable plaques of a patient as described in U.S. Pat. No. 7,351,421 to Sung et al., entitled Drug-Eluting Stent Having Collagen Drug Carrier Chemically Treated with Genipin,” incorporated herein by reference. Because the relatively low toxicity of genipin, it can be used in a slow releasing format, such as those described in the '654 application.

Epoxies

Epoxy compounds have reactive functional groups that are reactive with several functional groups found in proteins. Based on these reactivities, epoxies can be used to crosslink proteins, especially collagen, within tissue. For example, the use of epoxy polymers as a collagen crosslinking/stabilization agent for heart valve tissue is described in U.S. Pat. No. 5,080,670 to Imamura et al., entitled “Bioprosthetic Valve,” incorporated herein by reference. The compositions described in this patent include polyglycidyl ethers of polyglycerols having a polymerization degree of 1 to 3 as well as polyglycidyl ethers of polyols, and some specific examples of compositions include, for example, glycerol diglycidyl ether, glycerol triglycidyl ether, diglycerol tetragylcidyl ether and ethylene glycol glycidyl ether. Non-polymeric epoxy collagen crosslinking/stabilization agents are described in U.S. Pat. No. 5,880,242 to Hu et al., entitled “Nonpolymeric Epoxy Compounds for Cross Linking Biological Tissue and Bioprosthetic Grafts Prepared Thereby,” incorporated herein by reference. This patent describes compounds with a formula R₁—CH₂—O—X—O—CH₂—R₂, where X is a straight chain aliphatic hydrocarbon with either 4 or 5 carbon atoms, R₁ or R₂ or both are epoxy groups, and the other of R₁ or R₂ is an aldehyde group if it is not an epoxy group. A specific example of a nonpolymeric epoxy crosslinking/stabilization compound is 1,4-butanediol diglycidyl ether.

Epoxy amine polymer compounds are also suitable collagen crosslinking/stabilization agents. The use of these compounds as tissue, i.e., collagen, crosslinking/stabilization agents is described further in U.S. Pat. No. 6,391,538 to Vyavahare et al., entitled “Stabilization of Implantable Bioprosthetic Tissue,” incorporated herein by reference. An example of a suitable poly-epoxyamine compound suitable as a collagen crosslinking/stabilization agent is triglycidylamine, a triepoxy amine.

Triglycidylamine (TGA) is a highly polar, water soluble, polyepoxy-crosslinking agent. TGA appears to bind relatively irreversibly and has high reactivity towards sulfur-containing amino acids such as methionine. TGA has been observed to form a crosslinked tissue that is less susceptible to calcification relative to glutaraldehyde crosslinked tissue, and TGA does not alter tissue mechanical properties as much as glutaraldehyde treatment. The properties of TGA crosslinked tissue are described further in Van Wachem P B, Brouwer L A, Zeeman R, et al. J Biomed Mater Res 2000; 53(1): 18-27 and Connolly J M, Alferiev I, Clark-Gruel J N, et al. Am J Pathol 2005; 166(1): 1-13), both of which are incorporated herein by reference.

Epoxyamines are molecules that generally include both an amine moiety e.g. a primary, secondary, tertiary, or quaternary amine, and an epoxide moiety. In general, the epoxyamine compound can be a monoepoxyamine compound and/or a polyepoxyamine compound. In some embodiments, a polyepoxyamine compound has at least two epoxide moieties and possibly three or more epoxide moieties.

In some embodiments of epoxyamines, the epoxide ring can be separated from the nearest amino moiety by between 1 and 5 atoms, i.e., a C₁-C₅ branched or linear alkyl or substituted alkyl chain, such as the methylene group, which separates the epoxide ring and the tertiary amine moiety in TGA. Other chemical groups which can be interposed between the epoxide ring and the nearest amino moiety include, for example, branched or linear alkenyl chains, substituted alkyl chains, ring groups and aromatic groups. The epoxyamine compounds can have high reactivity with a variety of functional groups including, for example, alcohols, amines, sulfur containing functional groups, such as thiols and the like.

Azide Esters

Free carboxyl groups on collagen can be converted into acyl azide groups, which react with free amino groups on adjacent side chains to crosslink the collagen tissue. This crosslinking approach is described in Petite H, Duval J L, Frei V, et al. Biomaterials 1995; 16(13): 1003-1008, incorporated herein by reference. Diphenylphosphoryl azide is an azide that is commonly used in the activation of free carboxyl groups on collagen to react with free amino groups to achieve crosslinking/stabilization in collagen tissue. See, Petite H, Frei V, et al, J Biomed Mater Res 1994, 28, 159-165, incorporated herein by reference. Published results suggest that azide ester treated tissues have the same resistance as glutaraldehyde-treated tissues to chemical solubilization by cyanogen bromide and to enzymatic digestion by collagenase, as described in Petite H, Rault I, Huc A, et al. J Biomed Mater Res 1990; 24(2): 179-187, incorporated herein by reference.

### Elastin Stabilization Agents

Elastin is a protein constituent of connective tissue contributing to the elasticity and recoil of the tissue. Moreover, elastin can be abundant in connective tissue. Elastin is considered the most abundant extracellular matrix protein found in the aortic wall. Elastin polypeptide chains are naturally cross-linked together to form rubber-like, elastic fibers. Unlike collagen, elastin molecules can uncoil into a more extended conformation when the fiber is stretched and can recoil spontaneously when the stretching force is relaxed. Elastin degeneration in connective tissue pathology is generally believed to be caused by enzymes including elastase enzymes and matrix metalloproteinase (MMP) enzymes that can be secreted by vascular cells as well as by infiltrating inflammatory cells. While many aspects of the methods and schemes of various enzymes leading to elastin degradation remain unknown, in general, it is believed that most enzymes attack and bind the protein at a site away from the natural crosslinks.

As discussed earlier, it is known that extracellular matrix comprises compositions with specific mechanical properties such as fibrous components, collagen fibers and elastin fibers. The result of the crosslinking is added thermal dynamic stability of the elastin stabilized tissue. While the elastin stabilizing agents may have non-specific binding to other proteins to other tissue components, the compounds can be particularly effective with respect to their binding to elastin to provide desired stabilization of the tissue.

Elastin Degeneration within Aneurysms

The physiological characteristics of aneurysms are believed to involve degradation of arterial structural proteins including elastin and collagen, calcification, and overall destruction of arterial architecture. These changes in the tissue can result in loss of mechanical properties and progressive dilatation. Due to its insolubility, natural desmosine and isodesmosine crosslinks, and extremely long biological half-life, elastin is generally perceived to be resistant to degradation. However, there are a specific set of enzymes, matrix metalloproteinases (MMP, particularly MMP-2, MMP-9, and MMP-12), which are capable of degrading elastin. MMPs are involved in normal physiological processes such as bone remodeling, wound healing, and angiogenesis. However, abnormally high levels of MMPs have been identified in pathological processes in many vascular diseases, and appear to be significant contributors to the formation and progression of AAAs. This fact is underlined by consistent reports of severe elastin degradation within these aneurysmal tissues, as evidenced by heavy degeneration of the arterial architecture, decreased medial elastin content and disrupted or fragmented elastic lamellae. This degradation is particularly significant when one considers the inability of elastin to promptly revitalize itself (as evidenced by its nearly 70-year biological half-life), unlike some other relatively dynamic matrix components.

Furthermore, degradation of elastin results in the release of soluble elastin peptides. These peptides may not be passive by-products of the degradation process. In particular, it has been suggested that elastin peptides are active in stimulating protease production, chemotaxis, cellular proliferation, and various other biological activities. The release of elastin peptides can result in a cascade of even more matrix degradation, as it has been suggested that interactions between these peptides and smooth muscle cells increase expression of the elastin laminin receptor (ELR). This binding with ELR, a 67 kDa receptor found on a number of cell types, subsequently can result in the promotion of greater MMP synthesis. The use of luminally-perfused elastin peptides as an aneurysm animal model, which elicits elevated MMP levels and matrix degradation at the site of perfusion, is also consistent with suggested biological effects of these peptides with respect to contributions to further degradation of vascular tissue.

Degradation of connective tissue can be prevented or slowed through the stabilization of the elastin component of the tissue with a phenolic compound contacted with the tissue. This beneficial effect is described further in the '834 patent and copending '121 and '156 applications. In particular, it is believed that any of a number of natural and synthetic phenolic compounds can bind elastin and thereby protect elastin from degradation, for instance due to the action of elastin degrading enzymes.

Phenolic Compounds as Elastin Stabilization Agents

Phenolic compounds in some embodiments include, for example, any compound that comprises at least one phenolic group bound to a hydrophobic core. While not wishing to be bound by any particular theory, it is believed that interaction between the phenolic compound and elastin proteins involve both the hydroxyl group as well as the hydrophobic core of the molecules. In particular, it is believed that phenolic compounds can stabilize elastin proteins through both steric means and bond formation and thereby protect sites on the protein susceptible to enzyme-mediated (e.g., elastase or MMP-mediated) cleavage. Specifically, it is believed that hydroxyl groups of a phenolic compound can bind elastin multivalently, for instance via hydrogen bond formation with amino acid residues such as polar amino acid residues including methionine, glycine and proline, such that multiple proteins can interact with a single molecule to create a three-dimensional cross-link structure involving multiple elastin molecules. Moreover, in certain embodiments, the phenolic compounds of the present invention can have one or more double bonds, with which the phenolic compounds can covalently bind to the elastin, forming an even stronger and more stable protective association between the phenolic compound and the elastin of the connective tissue.

In addition, the large hydrophobic regions of the elastin protein, which are believed to contain sites susceptible to elastase-mediated cleavage, are also believed to contain sites of association between the hydrophobic core of the phenolic compound and the protein. Thus, the association between the phenolic compound and the protein molecules are believed to protect specific binding sites on the protein targeted by enzymes through the association of the protein with the hydrophobic core of the phenolic compounds and can also sterically hinder the degradation of the protein through the development of the large three dimensional cross-link structures.

Phenolic compounds of particular interest have a hydrophobic core and one or more phenol groups extending from the hydrophobic portion of the molecule. For instance, exemplary phenolic compounds can include, for example, flavonoids and their derivatives (e.g., anthocyanins, quercetin), flavolignans, phenolic rhizomes, flavan-3-ols including (+)-catechin and (−)-epicatechin, other tannins and derivatives thereof (such as tannic acid, pentagalloylglucose, nobotanin, epigallocatechin gallate, and gallotannins), ellagic acid, procyanidins, and the like. Phenolic compounds for use as elastin stabilizing agents can be synthetic compounds or natural phenolic compounds. For example, natural phenolic compounds can include those found in extracts from natural plant-based sources such as extracts of olive oil (e.g., hydroxytyrosol (3,4-dihydroxyphenylethanol) and oleuropein, extracts of cocoa bean that can contain epicatechin and analogous compounds, extracts of Camellia including C. senensis (green tea) and C. assaimic, extracts of licorice, sea whip, aloe vera, chamomile, and the like.

Suitable phenolic compounds can be tannins and derivatives thereof. Tannins can be found in many plant species. For example, the tea plant (Camellia sinensis) has naturally high tannin content. Green tea leaves are a major plant source of tannins, as they not only contain the tannic and gallic acid groups, but also prodelphinidin, a proanthocyanidin. Tannins are also found in wine, particularly red wine as well as in grape skins and seeds. Pomegranates also contain a diverse array of tannins, particularly hydrolysable tannins.

Tannic acid is a common naturally derived tannin. Tannic acid can interact with elastin and thus can stabilize the elastin component. At one time, however, tannic acid-containing preparations were suspected of causing hepatoxicity. This toxicity has since been primarily attributed to poor purity of the preparations and the inclusion of toxic gallic acid residues in the compositions. Accordingly, in one embodiment, the compositions used herein include high purity tannic acid, with little or no free gallic acid residue included in the compositions. For example, in one embodiment, the compositions can include less than about 5% free gallic acid residue in the preparation. In one embodiment, the compositions can include between about 1% and about 5% free gallic acid residue in the composition. The compositions can comprise an effective amount of pentagalloylglucose (PGG). PGG, which is the portion of a tannic acid molecule includes the hydrophobic core of tannic acid as well as multiple phenolic hydroxy groups, but does not posses the outer gallic acid residues and the hydrolyzable ester bonds associated with tannic acid. Thus, the possibility of release of free gallic acid residues over the course of a long-term application process can be prevented through utilization of a compound having no gallic acid residues, such as PGG, as the selected agent. A person or ordinary skill in the art will recognize that additional ranges of gallic free acid residues within the explicit ranges above are contemplated and are within the present disclosure.

### Therapeutic Compositions and Corresponding Treatment

As described herein, various therapeutic compositions can be used to treat tissue to achieved desired improvement in the treatment of aneurysm tissue. The therapeutic composition generally comprises a protein stabilization/crosslinking agent, such as an elastin stabilization agent and/or a collagen crosslinking agent. In particular, some therapeutic compositions comprise a collagen crosslinking agent, such as those described above. In some embodiments, a collagen crosslinking/stabilization agent can be used alone for the stabilization of blood vessel tissue with respect to mechanical properties and enzymatic degradation associated with an aneurysm or other degenerative diseases. In additional or alternative embodiments, the combined use of an elastin stabilizing agent and a collagen crosslinking/stabilization agent can provide a desirable degree of both mechanical stabilization of the tissue as well as the introduction of resistance to further protease and other degradation pathways. The elastin stabilizing agent and collagen crosslinking/stabilization agent generally can be applied sequentially or simultaneously to the tissue. For the simultaneous delivery to the tissue, the elastin stabilizing agent and the collagen stabilizing agent can be delivered in a single composition and/or through the delivery of a plurality of simultaneously delivered compositions. The relative amounts of the active agents as well as the treatment times can be adjusted appropriately to achieve the desired modification to the tissue. Rinsing agents are described below that can facilitate removal of a therapeutic compound at the end of treatment and/or to decrease residual cytotoxic effects of a therapeutic agent at the end of treatment.

In general, a therapeutic composition comprises a therapeutic agent supported in a suitable solvent. A therapeutic composition can optionally comprise additional agents as described further below. The concentration of a therapeutic agent of the therapeutic composition can be in the range of about 0.0001% to about 25% (w/v). In some embodiments, the concentration can be in the range of about 0.005% to about 10% (w/v). In some embodiments, the concentration can be in the range of about 0.01% to about 1% (w/v). A person of ordinary skill in the art will recognize that additional ranges of concentration within the explicit ranges above are contemplated and are within the present disclosure.

In general, the agents described herein can be provided as a suitable therapeutic composition. In one embodiment, the therapeutic compositions are used to stabilize connective tissue in vivo. Accordingly, in such embodiments, biocompatibility and cytotoxicity of the agents is considered in preparation of therapeutics including the disclosed agents and compositions, although localized delivery of the agents can mitigate these properties. In particular, through the localized delivery of the therapeutic agents, the agents can have a greater cytotoxicity than can be tolerated for systemically delivered agents. Thus, there may be a balance of potential cytotoxicity and the efficacy of the agents to stabilize the tissue matrix.

It is believed that the tissue architecture and cell viability in the aneurysmal areas are deteriorating by the time that the aneurysms are detected. Because of the deteriorating condition of aneurysmal tissues, certain toxicity in the therapeutic treatment agent can be tolerated based on a need to stabilize the tissue. In general, a therapeutic agent containing an effective amount of the collagen crosslinking/stabilization agent can be delivered by a device, such as devices disclosed in the '833 application, to reduce the systemic toxicity and potential side effects. Furthermore, the cytotoxicity associated with glutaraldehyde or other aldehyde based collagen crosslinking/stabilization agents can be reduced with appropriate rinsing agents described further below. Alternatively, for compositions with low toxicity such as genipin, slow release formulation can be used to deliver the crosslinking/stabilization agent, such as the formulations described in the '654 application.

Once delivered to the targeted blood vessel by any suitable method, the composition can access and then stabilize the connective tissue of the vessel. For instance, when delivered to the connective tissue from the lumen of a blood vessel, the composition disclosed herein may penetrate the endothelium of the vessel wall to contact the elastin and/or collagen of the connective tissue and stabilize the structure architecture.

Therapeutic compositions can optionally comprise additional agents, in addition to agents that stabilize collagen and/or elastin. Such additional agents can be active agents, providing direct benefit to the tissue, or may be supporting agents, improving delivery, compatibility, or reactivity of other agents in the composition. For example, in one embodiment, the compositions can be combined with any of a number of possible lipid-lowering medications so as to prevent the development of calcified lipid deposits or arteriosclerosis plaques that can often be found in conjunction with aneurysm formation. Lipid lowering medications may include, for example, statins, niacin and fibrates.

The therapeutic composition can comprise one or more buffers. For example, a composition may be formulated with inclusion of purified water, saline and a biocompatible buffer, such as phosphate buffers, borate buffers, HEPES, PIPES, and MOPSO. In some embodiments, a composition may be formulated to have a pH of between about 5.5 and about 7.4. In one embodiment, the composition has a pH of about 6.2.

Compositions for parenteral delivery, e.g., via injection or localized delivery in the vasculature with the devices herein, can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. In addition, the composition can contain minor amounts of auxiliary substances such as wetting or emulsifying agents, pH buffering agents and the like that can enhance the effectiveness of the phenolic compound. These compositions may also contain adjuvants such as preservatives, wetting agents, emulsifying agents, surfactants and dispersing agents.

Prevention of the action of microorganisms may be provided by the inclusion of various antibacterial and antifungal agents such as paraben, chlorobutanol, phenol, sorbic acid and the like. It may also be desirable to include isotonic agents such as sugars, sodium chloride and the like.

In some embodiments, the compositions can include pharmaceutically acceptable salts of the components therein, e.g., those that may be derived from inorganic or organic acids. Pharmaceutically acceptable salts are well known in the art. For example, S. M. Berge, et al. describes pharmaceutically acceptable salts in detail in J. Pharmaceutical Sciences (1977) 66:1 et seq., which is incorporated herein by reference. Pharmaceutically acceptable salts include the acid addition salts that are formed with inorganic acids such as, for example, hydrochloric or phosphoric acids, or such organic acids as acetic, tartaric, mandelic and the like. Salts formed with free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Representative acid addition salts include, but are not limited to acetate, adipate, alginate, chloride, sulfate, citrate, aspartate, benzoate, benzenesulfonate, bisulfate, butyrate, camphorate, camphorsulfonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others.

### Rinsing Compositions

In some embodiments, it is desirable to rinse an isolated portion of a blood vessel following completion of a treatment with a therapeutic composition. Rinsing compositions can comprise a simple rinse that is intended to remove unreacted therapeutic compositions through dilution and physical fluid exchange. In some embodiments, a rinse agent can comprise a composition that decreases any residual toxicity associated with the treatment agent. In some procedures, a plurality of rinsing agents can be used in a selected order with respect to each other and with respect to the application of therapeutic compositions. The volume of rinsing agent can be selected to achieve the desired rinsing effect.

In principle, a rinsing agent can be sterile deionized or distilled water. But pure water can be harsh on tissue, so generally it would be desirable to use saline, e.g. water with sodium chloride, at or near physiological ionic strength, e.g., about 0.9% w/v NaCl. Saline can optionally include a suitable buffer. Physiologically acceptable buffers are known in the art, such as phosphate salts, carbonate salts, TRIS (tris(hydroxymethyl)methylamine), MOPS (3-(N-morpholino)propanesulfonic acid), MES (2-(N-morpholino)ethanesulfonic acid) or tricine (N-tris(hydroxymethyl)methylglycine). Suitable sterile salines are available commercially for intravenous use that can be adapted as rinsing agents.

Following treatment with aldehyde crosslinking agents, the unreacted free aldehyde groups on the treated tissue can be further treated with compounds that are reactive amines such as lysine or glutamine, thus lessening the impact of potential aldehyde cytotoxicity. See, Simionescu A, et. al., R. J Biomed Mater Res 1991; 25(12): 1495-1505 and Jorge-Herrero E, et al. Biomaterials 1996; 17(6): 571-575, both of which are incorporated herein by reference. Other amines that can be used including other amino acids as well as diamines, such as 1,6 hexane diamine or N,N,N′,N′-tetramethyl diamine. Alternatively, aldehyde groups from a collagen crosslinking/stabilization agent can be quenched with organic or inorganic agents disclosed in U.S. Pat. No. 6,471,723 to Ashworth et al., entitled “Biocompatible Prosthetic Tissue,” incorporated herein by reference. Ashworth et al. discovered that improved reduction in cytotoxicity could result from the use of a plurality of toxicity reducing agents.

In addition to amines, suitable toxicity reducing agents can include, for example, inorganic sulfur-oxygen containing anions, organic sulfates, ammonium salts and surfactants. Suitable inorganic sulfur-oxygen anions include, for example, sulfate anions SO₄⁻², thiosulfate anions S₂O₄⁻², and protonated forms thereof, such as bisulfate HSO₄⁻. Suitable organic sulfates include, for example, aliphatic sulfates, such as methyl sulfate CH₃O₄⁻, dimethyl sulfate (CH₃)₂O₄S and dodecyl sulfate CH₃(CH₂)₁₁O₄S⁻ as well as protonated forms thereof. Ammonium cations can be introduced, for example, as ammonium chloride, ammonium hydroxide or other suitable ammonium salt.

Suitable surfactants include, for example, nonionic surfactants, such as aliphatic fatty acid esters, polypropyleneglycol fatty acid esters, e.g., propyleneglycol monostearate, and glycerol fatty acid esters, e.g., glycerol monostearate. Other suitable nonionic surfactants include, for example, polyalkylene ethers, such as polyethylene lauryl ether, polyoxyethylene oleyl ether, polyoxyethylene cetyl ether, and polyethylene glycol p-isooctyl phenyl ethers, such as Triton™ X-100 and the like. Furthermore, suitable nonionic surfactants include, for example, polyoxyethylene sorbitan esters that include, for example, polyoxyethylene compounds in an ether linkage with sorbital and an ester linkage with a fatty acid, such as Polysorbate 80 or polyethylene (2) sorbitan monooleate, which is sold under the trade name Tween® 80. Other nonionic surfactants are used for medical applications, which are suitable for rising agents.

Toxicity reducing agents are generally dissolved into an aqueous solution for use. The solution can be buffered, for example, using a physiological buffer described above. In some embodiments, the toxicity reducing agents can be included in a concentration from about 0.005 molar (M) to about 3 M, and in further embodiments from about 0.01M to about 1M. A person of ordinary skill in the art will recognize that additional ranges of concentrations within the explicit ranges above are contemplated and are within the present disclosure. As noted above, it can be desirable to include two different classes of toxicity reducing agents within the classes of agents described above. In particular, Ashworth et al., above, suggest the use of a combination of an amine, a sulfate, ammonium ions and a surfactant.

### Delivery Vehicles

As described herein, therapeutic compositions can be delivered in various formats. In some formats, the therapeutic agents are delivered in a format for rapid efficacy since the treatment time is limited. In other embodiments, the therapeutic composition can be provided over more extended periods of time. The delivery of therapeutic agents using a delivery vehicle, such as for delayed release, is described further in the '654 application. In some embodiments, the method can comprise use of timed release or sustained release delivery vehicles. Such systems can be desirable, for instance, in situations where long term delivery of the agents is appropriate and desired.

The dosage of the disclosed treatment agents can depend on the disease state or particular condition being treated and other clinical factors such as condition of the patient and the size and design of the device. In addition, the disclosed treatment agents can be administered in conjunction with other forms of therapy, e.g., surgical endovascular stent. The combination of the two treatments can reduce the risk of future expansion of the aneurysm and risk of graft migration at the aneurysm neck.

In some embodiments, the delivery devices described herein can be used for delivery of a composition comprising collagen stabilization agent and/or elastin stabilization agent that are targeted to a specific site, such as to a diagnosed aneurysm in vivo, using a less invasive procedure to provide delivery of the treatment agent locally from the interior of the vessel. Percutaneous entry into blood vessels and corresponding delivery technologies for catheter use for vascular procedures suitable for introducing the devices described herein are generally known to those of skill in the art, and can be adapted for use with the devices for the delivery of therapeutic compositions described herein. Delivery of the therapeutic agent using the delivery devices can be supplemented with the use of a stent, which may or may not further comprise a drug coating. U.S. Pat. No. 6,979,347 to Wu, et al., incorporated herein by reference, describes an apparatus and associated method for delivering a therapeutic substance through a coating on a stent, such as the genipin. For example, the genipin compound, or a composition thereof, can be deposited into grooves on a stent using conventional spray or modified dip techniques.

According to one embodiment, a sustained-release matrix can comprise materials, usually polymers, which are degradable by enzymatic or acid/base hydrolysis or by dissolution. Once located at or near the target tissue, e.g., inserted into the body, for instance in the form of a patch or a stent such as those further described below, such a matrix can be acted upon by enzymes and body fluids. The sustained-release matrix can be chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Possible biodegradable polymers and their use are described, for example, in detail in Brem et al. (1991, J. Neurosurg. 74:441-6), which is hereby incorporated by reference in its entirety. In some embodiments, the composition can be loaded in a drug delivery vehicle via encapsulation, coating, infusion, or any other loading mechanism, such as those known in the art.

In one embodiment, the disclosed agents can be targeted to connective tissue by use of a hydrogel delivery vehicle. Hydrogels herein refer to polymeric matrices that can be highly hydrated while maintaining structural stability. Hydrogels are hydrophilic polymers that do not dissolve in aqueous solution generally as a result of crosslinking. Hydrogel delivery vehicles can include, for example, natural polymers such as glycosaminoglycans, polysaccharides, proteins, and the like, as well as synthetic polymers, as are generally known in the art. Pluronic™ polymers generally comprise polyoxy-propylene/polyoxyethylene block copolymers. Thus, hydrogels from the crosslinking of these block copolymers and similar compositions can be referred to as Pluronic™ hydrogels.

Polymeric particles for drug delivery generally include, for example, biocompatible polymers and may or may not be spherical. The polymeric particles generally have an average particle diameter of no more than about 5 microns, in further embodiments no more than a micron and in additional embodiments no more than about 250 nanometers, where the diameter is an average dimension through the particle center for non-spherical particles. The delivery of drugs using nanoparticles and microparticles is described further in published U.S. Patent application 2006/0034925 to Au et al, entitled “Tumor Targeting Drug-Loaded Particles,” incorporated herein by reference.

PGG formulations have been shown to form a gel under certain conditions. The conditions, such as concentration, during formation of the gel influence the resulting gel properties. In some embodiments, the PGG gel can be formulated to dissolve around 37° C., the body temperature of a patient. Additionally or alternatively, PGG can be formulated as a gel that remains its gel form at around 37° C. or higher temperatures. The gel forms PGG can be used as drug delivery vehicle, for example, a slow release delivery vehicle, with properties adjusted as desired. The gel form PGG can also be used in combination with other delivery systems such as hydrogel and/or poly(lactic-co-glycolic acid) nanoparticles to provide release profiles for short or extended period (28 days).

The treatment approaches described herein provide considerable flexibility. For example, more toxic treatment compositions can be delivered using approaches in which the treatment composition is left in the vessel for a limited period of time, while another therapeutic composition that is less toxic can be delivered with a delivery vehicle for more extended contact with the tissue to be treated. Treatment procedures are described in more detail below.

Treatment with therapeutic agents can be combined with mechanical stabilization. In particular, a perivascular girdle wrap can be placed over the exterior of the aneurysm to provide mechanical stabilization along with the chemical stabilization. In some embodiments, the delivery systems described herein can be associated with a perivascular girdle wrap. For example, the delivery systems can be coated along the interior of the wrap and/or embedded in the material of the wrap. The wrap provides a close contact to the aneurysm site for consistent drug release. In these embodiments, the girdle wrap physically strengthens the vasculature at the aneurysm site to prevent it from bursting while the elastin stabilizing agents can act to stabilize and strengthen the tissue of the vessel along with inhibiting further degradation of the vessel at the location. The wrap can be formed from biocompatible polymers, such as polyesters, that can be formed into woven or non-woven fabrics.

### Devices for the In Vivo Localized Vascular Delivery of Therapeutic Compositions

In some procedures it is desirable to deliver therapeutic compositions to an isolated section of a blood vessel. Through the isolation of a section of a blood vessel, greater control can be maintained over the treatment process and any toxicity issues can be better controlled. Further, sequential steps can be performed more directly on the same section of tissue. Thus, sequential collagen stabilization and elastin stabilization steps can be performed, rinses can be performed, and the like. Devices have been developed to isolate section of blood vessels with the capability of exchanging fluids with the isolated portions of the vessel. In particular, percutaneous procedures can provide access to the interior of blood vessels. To provide more effective treatment of a selected region of the interior of a blood vessel, regions of the blood vessel can be isolated for treatment using appropriate device. In particular, the delivery devices described in the '833 application and the '624 patent provide for the introduction into a blood vessel using less invasive procedures.

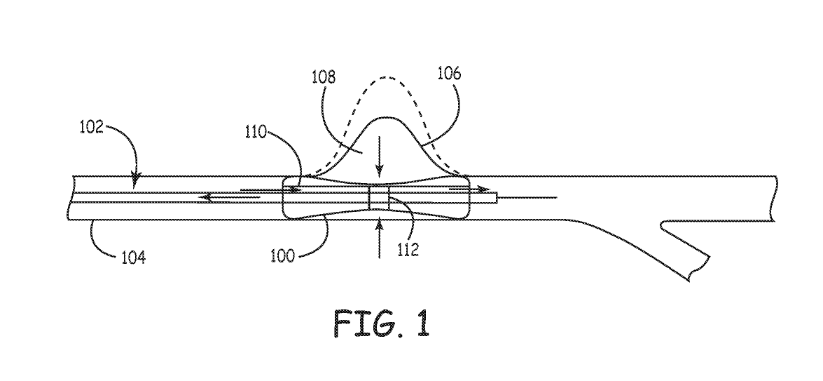

With respect to devices described in the '833 application, a sealing element of the device can be positioned near an aneurysm or other location for treatment within a vessel such that when deployed the sealing element isolates the selected section of the vessel. In general, as illustrated in FIG. 1, a representative device 102 comprises a shaft, a sealing element 100 and flow lumen(s) that provide flow passages between locations at or near the distal end to locations at or near the proximal end. The one or more flow lumens extend through the shaft to a fluid exchange portion 112 to provide for delivery to and/or removal of fluids from the isolated volume 108 of the vessel 104. The sealing element 100 is configured to isolate the volume 108 against the wall of the vessel 104 at a selected location such that the aneurysm 106 or other region of interest can be accessed for localized delivery of a treatment fluid. Generally, the device further comprises a passageway 110 that provides for vessel flow past the sealing device. The device optionally can comprise a guide lumen for the over-the-wire or rapid exchange interface of the device with a guidewire. While the device can be effective for the treatment of an aneurysm, the device can be used in other circumstances for the localized treatment of a portion of a blood vessel.

The procedures generally vary in some respects based on the nature of the sealing element. For example, devices with two or more flow lumen can provide for aspiration through one lumen and delivery of fluids through another lumen, while a single lumen device requires sequential approaches. In some embodiments, additional steps of delivering and removing liquids from the isolated region can be performed if desired, such as for the sequential contact with stabilization fluids. In one embodiment, the step of delivering the therapeutic composition is repeated with a different composition, wherein the delivery of one therapeutic composition comprises the delivery of a collagen stabilization composition and the delivery of the other therapeutic composition comprises the delivery of an elastin stabilization composition. The order of the delivery agents can be selected as desired.

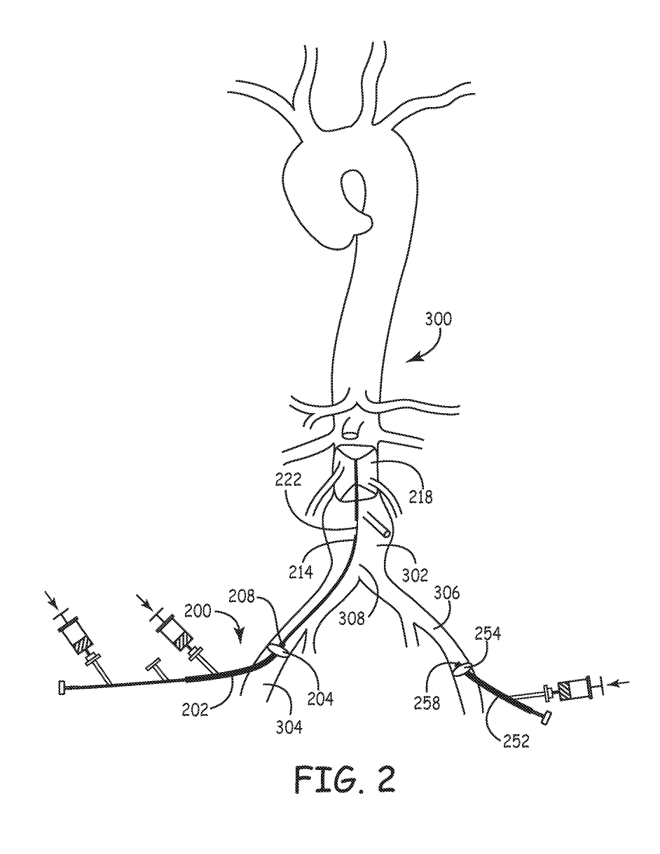

A device for sealing two ends of a selected segment of a vessel with an adjustable length is described in the '624 patent, with a representative embodiment shown in FIG. 2. The device generally comprises an introducer sheath 202 and a sealing catheter 214. The sealing catheter 214 can comprise an extendable element 218 and fluid exchange opening 222. The extendable element 218 of the catheter generally has a low profile delivery configuration and a deployed, extended configuration that contacts and seals against the wall of the vessel. In some embodiments, the sealing element 218 has concave ends that facilitate good sealing against the walls of the vessel while applying modest amounts of force against the vessel wall. The introducer sheath 202 can comprise an extendable element 204 that is mounted at or near the distal end of the introducer sheath. The extendable element 204 of the introducer sheath 202 generally has a low profile delivery configuration and a deployed, extended configuration that pushes against and seals the walls of the vessel. In some embodiments, the catheter lies coaxially inside a main lumen of the introducer sheath. The gap 208 between the main lumen of the introducer sheath and the catheter can also optionally function as a fluid exchange lumen. In the combined device, the sealing element 204 of the introducer sheath 202 is located at the proximal end of the sealed vessel segment and the sealing element 218 of the catheter 214 is at the distal end of the sealed vessel segment.

The two extendable elements 218 and 204 of the combined device are movable relative to each other. The volume and location of the isolated vessel portion therefore can be adjusted based on the relative positions of the two extendable elements. When treating aneurysm 302 for example, one of the extendable elements 204 can be placed in left femoral artery 304 close to the vessel access point where the device enters the vessel while the other extendable element 218 can be placed in aortic artery 300 beyond the site of aneurysm 302 or a significant portion thereof. Another introducer sheath 252 can be used to have extendable element 254 be placed in the right femoral artery 306. When the three extendable elements are deployed, an isolated volume 308 encompassing the aneurysm 302 is created between the three extendable elements 204, 218, and 254. The extendable element placed close to the vessel access point can be called the proximal extendable element, which may be part of an introducer sheath 202 or 252. The extendable element placed beyond the site of aneurysm can be called the distal extendable element 218, which may be carried on a sealing catheter 214. Improved designs of the extendable element of the sealing catheter such as a sealing balloon for placement in the aorta provide for good sealing properties with reduced risk of damaging potentially vulnerable vessel tissue. The sealing introducer sheath provides considerable versatility to the procedure, and the sealing introducer sheath can be effectively used without the sealing catheter component for the performance of procedures, for example, in the right femoral artery described above.

An alternative device for isolating a section of a blood vessel is described in published U.S. patent application 2001/0029349A to Leschinsky, entitled “Method and Apparatus for Treating Aneurysm,” incorporated herein by reference.

### Treatment Procedures

In general, the therapeutic compositions are delivered to the vicinity of a connective tissue site to be stabilized. In some embodiments, the connective tissue is a portion of a blood vessel, such as a portion of a blood vessel exhibiting an aneurysm. The therapeutic compositions disclosed herein can be delivered perivascularly, endovascularly, or a combination thereof. Perivascular procedures can be performed in an open format or using a laparoscope or the like to reduce the invasiveness of the procedure. Endovascular procedures can be desirable due to the reduced invasiveness of the procedure and a correspondingly reduced recovery process for the patient.

Known perivascular drug delivery technologies include those described by Chen, et al. (U.S. Patent Application Publication No. 2005/0079202, entitled “Implantable Elastomeric Depot Compositions and uses Thereof”) and Nathan (U.S. Patent Application Publication No. 2003/0228364, entitled “Amphiphilic Polymers for Medical Applications”), both of which are incorporated herein by reference. These exemplary perivascular delivery systems comprise a polymeric delivery vehicle that can be injected or directly placed, for instance via surgery, at a particular location so as to provide controlled release of therapeutic composition encapsulated or otherwise loaded therein over a period of time. Alternatively, a perivascular girdle or wrap with embedded therapeutic composition may be applied to the exterior of aneurysm to both mechanically and physically strengthen the aneurysm vessel.

In general, any reasonable endovascular technique can be used to deliver the therapeutic composition. Some endovascular drug delivery methods are known in the art. For example, DiCarlo, et al. (U.S. Pat. No. 6,929,626, entitled “Intraluminally Placeable Textile Catheter, Drain and Stent,” incorporated herein by reference) describe an intraluminally placeable tubular device that can be located within the lumen of a blood vessel and coated or otherwise loaded with therapeutic compositions described herein. For example, the tubular member can comprise yarns interconnected in a pattern defining opposed interior and exterior textile surfaces. At least one of the textile surfaces is the body fluid-contacting luminal surface or the body lumen-contacting exterior surface. However, endovascular procedures using devices to isolate portions of the interior of the blood vessel, as described above, provide considerable versatility with respect to the delivery of therapeutic agents, rinse agents and the like in a selected order.

Devices such as those described in the above section can be utilized to isolate a section of the blood vessel. The isolated section of the blood vessel may contain aneurysms such as those depicted in FIGS. 1 and 2. The isolated vessel section can be aspirated to relieve pressure buildup and generally clear the vessel section for treatment. In the case of aneurysm, the volume of the expanded aneurysm wall may be reduced because of the reduced pressure inside the vessel when the isolated section is aspirated. FIG. 1 for example, shows aneurysm 106 is reduced in size once the isolated section is aspirated. The reduced sized aneurysm, when treated with crosslinking agents described herein may maintain at least part of the reduction of the expansion of the vessel wall due to the strengthening of the vessel tissue.

The devices that are designed to isolate the section of the interior of the blood vessel generally provide the capability for the delivery and removal of fluids from the isolated section of the vessel. Due to the relative incompressibility of liquids, it is generally desirable to remove fluids through suction, i.e., aspiration, prior to and/or simultaneously with the delivery of fluids. Also, the removal of fluids prior to and/or simultaneously with the delivery of fluids reduces dilution of the delivered fluids. Furthermore, suction can be useful for the removal of fluids at the completion of a treatment step so that most of the remaining therapeutic composition is not released systemically at the end of the procedure. Suction can also facilitate the partial remolding of an aneurysm shape through the reduction of pressure in the vessel that can allow some relaxation of the expanded vessel. Based on these principles, a procedure within the isolated section of blood vessel generally comprises one or more aspiration steps. A particular aspiration step can be performed simultaneously with fluid delivery, separately from fluid delivery or overlapping in any appropriate way with fluid delivery. If the patient's blood is removed in an initial step of a treatment process, some or all of this blood can be returned at the end of the procedure.

Using a medical device to isolate a section of the blood vessel for isolated endovascular treatment, the isolated section of the blood vessel can be optionally pretreated to prepare the tissue for more effective treatment and to better expose the tissue of the blood vessel for easy access by the therapeutic agents. For instance, a thrombolytic agent can be applied to the isolated section of the blood vessel to break up thrombus. Through the delivery to an isolated section of the vessel a thrombolytic agent can be delivered safely at a higher concentration, and after a desired treatment period, at least a substantial portion of the remaining thrombolytic agent can be safely removed. Suitable thrombolytic agents to remove thrombus or calcification can include, for example, tissue plasminogen activator (tPA) or urokinase, or a mild acid or anti-calcification enzymes such as osteopontin to resorb calcific plaque, or a combination thereof. Other suitable thrombolytic (antithrombic) agents include, for example, heparin, aspirin, adenosine diphosphate receptor antagonists, and Gp IIb/IIIa inhibitors. The isolated section of the blood vessel provides opportunities for therapeutic compositions that have relatively high toxicity that are generally kept away from systemic circulation. After aspiration removal of the pre-treatment agents, the isolated vessel section maybe rinsed with saline and/or buffer to remove leftover or unbound pre-treatment agent.

Using the devices to isolate the section of the blood vessel, therapeutic compositions comprising collagen crosslinker, elastin crosslinker, or a combination of collagen and elastin crosslinker can be administered to the isolated vessel section to allow crosslinking/stabilizing to occur. In some embodiments, the isolated section of the vessel can be treated with the therapeutic composition for about 1 min to about 4 hours. In further embodiments, the treatment can last about 2 min to about 3 hours. In some other embodiments, the treatment can last about 5 min to 2 hours. A person of ordinary skill in the art will recognize that additional ranges of time within the explicit ranges above are contemplated and are within the present disclosure. If the device provides for flow of blood past the isolated section of the vessel, the treatments in principle can be performed for longer periods of time if the isolated section of the vessel is appropriately positioned.

The aspiration step and the delivery of the therapeutic composition steps of the procedure can be repeated and the therapeutic composition delivered for each repeat can be the same or different compositions. In some embodiment, the therapeutic composition delivered comprises collagen crosslinker. In other embodiment, the therapeutic composition delivered comprises elastin crosslinker. In another embodiment, the therapeutic composition comprises both collagen crosslinker and elastin crosslinker/stabilizer. In one embodiment, as shown in FIG. 1, the flow in the vessel is maintained through a by pass channel 110 of the delivery device 102 while treatment is performed.

Rinses can be applied in between treatment steps to eliminate unwanted interaction between different treatment compositions as well as to remove unreacted treatment compositions. In some embodiment, rinses may contain quenching agents, such as amino acid or serum albumin, to reduce the toxicity of the therapeutic compositions after treatment. Rinses with toxicity reducing agents can be particularly desirable for embodiments based on collagen crosslinking with aldehydes, and appropriate toxicity reducing agents are described in detail above.

Some collagen crosslinking agents have greater relative cytotoxicity relative to some elastin crosslinking/stabilization agents. Therefore, it can be desirable to use different approaches for the delivery of these agents. For example, the collagen stabilization agents can be delivered to a localized section of the blood vessel and removed after a suitable period of time. Thus, for example, glutaraldehyde can be delivered into a section of blood vessel with the excess removed following completion of the treatment. One or more rinses with or without toxicity reducing agents can be applied following completion of the contact with the aldehyde. Then, the elastin stabilization agent can be delivered either in a direct approach or with a controlled release delivery vehicle. If the elastin stabilization agent is delivered with a controlled or delayed release delivery vehicle, this composition can be left in the vessel for more extended period of time with gradual release, and some of the delivery vehicles naturally decompose after a reasonable period of time. The delivery vehicle can be just coated onto the wall of the vessel and/or the delivery vehicle can be associated with a mechanical structure, such as a stent or the like, to stabilize the delivery vehicle in the vessel.

Wu, et al. (U.S. Pat. No. 6,979,347, entitled “Implantable Drug Delivery Prosthesis,” incorporated herein by reference) describe an apparatus and associated method for delivering a therapeutic composition, such as described herein, to a vascular lumen. Specifically, an implantable prosthesis, such as a stent, can be utilized that has grooves or trenches formed thereon. The grooves are formed on specific regions of the stent struts to increase the flexibility of the stent. The grooves also provide a location for carrying the therapeutic composition for delivery from the device following implantation. For example, the therapeutic composition can be deposited directly into the grooves using conventional spray or modified dip techniques.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein to the extent that the incorporated material is not contrary to any of the explicit disclosure herein.

### Examples

All reagents are from Sigma unless specified. For example, PGG solid/powder is obtained from Ajinomoto OmniChem (Mont-St-Guibert, Belgium). Glutaraldehyde (Glut) is obtained from Alfa Aesar (Ward Hill, Mass.), supplied as 25% (w/v) aqueous solution. Unless otherwise specified, saline (0.9% w/v or 9 g/L) was used as control as well as solvent to make the PGG and Glut testing samples throughout the examples.

### Example 1

### Opening Angle Test to Determine Mechanical Property of the Treated Tissue

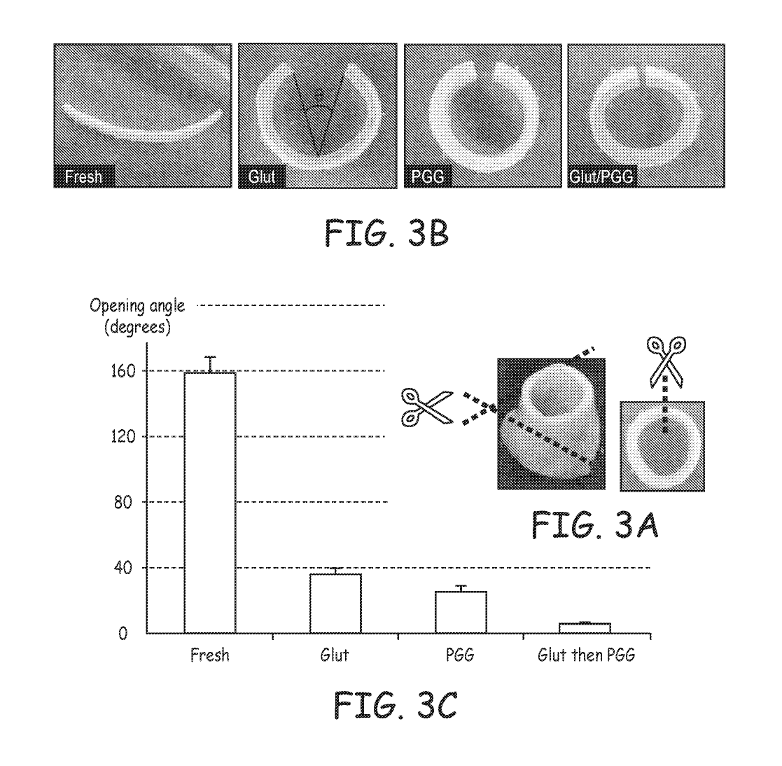

Porcine aorta obtained from a USDA approved slaughterhouse were cut transversely into ring segments approximately 1 cm in height as shown in FIG. 3A. The rings were left untreated (fresh sample) or treated with glutaraldehyde (Glut), PGG, or Glut then PGG (Glut/PGG). Glut treatment was performed with 0.6% (w/v) Glut for 1 day, and then 0.2% (w/v) Glut for 7 days, all done at room temperature; PGG treatment was performed with 0.15% (w/v) PGG for 4 days at 37° C. Glut/PGG treatment was performed with 0.6% (w/v) Glut for 1 day, and then 0.2% (w/v) Glut for 7 days at room temperature followed by 0.15% (w/v) PGG for 4 days at 37° C.

After treatments were completed, the aortic rings were immersed in water with the cross section of the aorta facing upward, allowing free movement of the aortic tissue. The aortic rings were cut once in the radial direction, as shown in FIG. 3A and allowed to “relax” and open for 15 minutes under water, and then digitally photographed. The photographs were shown in FIG. 3B. The digital photographs were then used to calculate the opening angle of each aortic ring graphically using Adobe Photoshop 7.0. The opening angle of each aortic ring was compared in graphical format in FIG. 3C. As shown in FIG. 3C, while the fresh sample has an opening angle of close to 160 degrees, the treatment with Glut has significantly altered the mechanical property of the aortic ring as shown by the significant reduction of the opening angle to close to 40 degrees. Treatment with PGG alone reduced the opening angle even further relative to glutaraldehyde treatment alone. The most significant reduction is seen in the treatment by Glut then PGG with an opening angle of less than 10 degrees.

### Example 2

### Tissue Resistance to Collagenase Degradation after Treatment

Tissue resistance to collagenase degradation after treatment with various reagents is discussed. Specifically, samples of porcine aortic wall were either left untreated (fresh) or treated with Glut alone or Glut followed by tannic acid (TA). Glut treatment was performed with 0.6% (w/v) Glut for 1 day, and then 0.2% (w/v) Glut for 7 days at room temperature; Glut/TA treatment was performed with 0.6% (w/v) Glut for 1 day, and then 0.2% (w/v) Glut for 7 days at room temperature followed by 0.15% (w/v) TA for 4 days at 37° C. The treated samples were rinsed 3 times (1 hour each) in 100 mL water, and lyophilized to record dry weight. Samples with the amount of ˜15 to 25 mg dry weight were immersed in 1.2 mL of type I collagenase (150 U/mL) dissolved in 50 mM Tris buffer, 10 mM CaCl₂, pH 7.4, and incubated at 37° C. with orbital shaking at 650 rpm for 24 hours. Following this exposure to collagenase, samples were centrifuged (10000 rpm, 10 minutes, 4° C.), individually rinsed three times in 1 mL water, lyophilized to obtain dry weight after collagenase, and the percent of digested tissue was calculated.

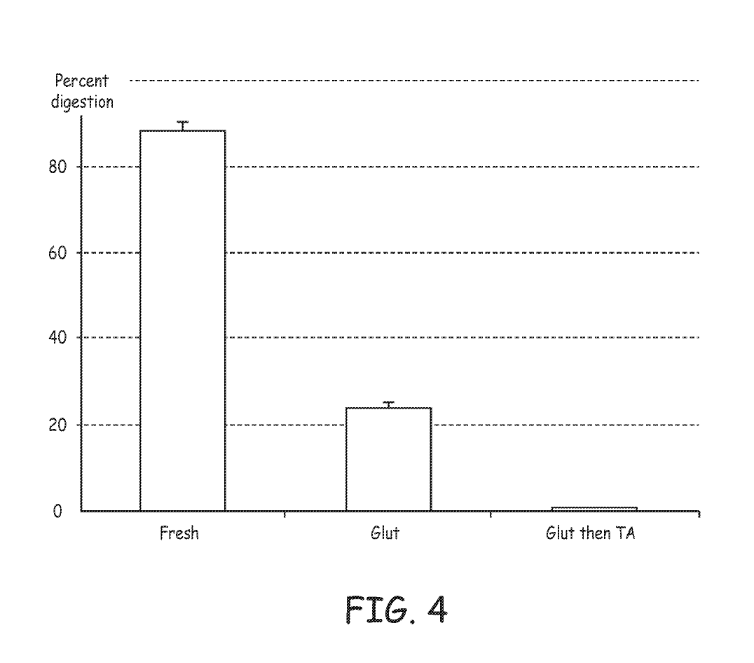

The percentage of tissue digestion was compared in graphical format in FIG. 4. As shown in FIG. 4, while over 85% of the fresh sample has been digested in this accelerated model, the percentage of the sample digested was reduced to slightly over 20% after treatment with Glut. Mass loss values for aorta treated with Glut and TA were essentially zero, suggesting that tannins may even enhance the ability of Glut to protect collagen from enzymatic degradation.

### Example 3

### Thermal Denaturation Temperature of Treated Tissues

The thermal denaturation temperatures (Td), common indicators of collagen crosslinking density, were measured in samples from treatment groups using a differential scanning calorimeter (DSC) (Perkin-Elmer DSC 7; Boston, Mass.). The samples were treated under the conditions outlined in Example 2. The treated aortic wall samples (approximately 2 mm×2 mm) were sealed in aluminum pans, heated at a rate of 10° C. per minute from 20° C. to 110° C. Td was determined as the temperature measured at the endothermic peak. This observed endothermic peak occurs at the temperature where collagen fibers unravel or denature, resulting in a measurable release of energy. Therefore, a higher denaturation temperature correlates into improved collagen crosslinking. The Td data from the samples are recorded in Table 1. According to the data in Table 1, fresh untreated sample has Td that is significantly lower than the Glut treated sample, indicating significant increase of degree of collagen crosslinking. The additional treatment with TA following the Glut treatment resulted in a further slight increase in Td.

TABLE 1

| | Thermal Denaturation |
|---|---|
| Treatment Group | Temperature (T_{d}) |
| Fresh | 68.37 ± 0.67° C. |
| Glut | 90.43 ± 0.27° C. |
| Glut then TA | 92.92 ± 0.93° C. |

### Example 4

### Tissue Resistance to Elastase Degradation after Treatment

This example involves model results based on in vitro treatment of tissue samples for period of time that are realistic for in vivo applications. Specifically, tissue resistance to elastase degradation after treatment with various reagents is discussed. Specifically, porcine carotid arteries were treated with saline (control, for 20 minutes), Glut (for 20 minutes), PGG (for 20 minutes), or a combination of the two (Glut+PGG for 20 minutes, or Glut for 10 minutes followed by a separate incubation with PGG for 10 minutes). Concentrations of the reagents used were 0.6% (w/v) for Glut, 0.15% (w/v) for PGG and 9 g/L for physiological saline.

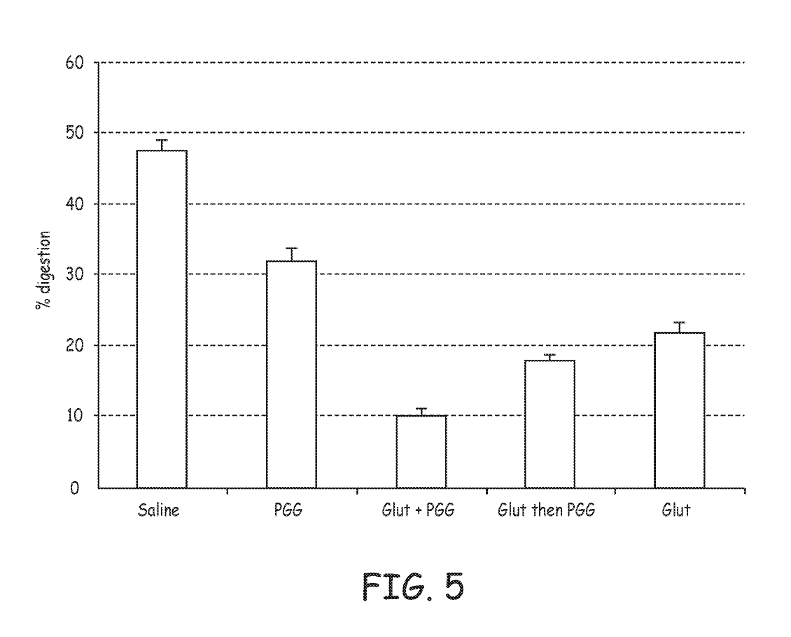

The treated tissue was then exposed to an in vitro elastase digestion assay to subject the treated tissue to digestion for 24 hrs. Briefly, tissue samples were incubated individually with 1 mL of 3 units/mL porcine pancreatic elastase (Elastin Products Company; Owensville, Mo.) dissolved in 100 mM Tris buffer, 1 mM CaCl₂, 0.02% NaN₃, pH 7.8. Incubation with the elastase was conducted for 24 hrs at 37° C. with orbital shaking at 500 rpm. The percentage digestion of the arteries was measured by mass loss after the assay and results are shown in FIG. 5. Because the values shown are percentage of digestion, the lower the value, the better the reagent used performed in resisting elastase degradation. Individually, Glut and PGG each slightly improved the resistance of the tissue to degradation as compared to saline controls. When Glut and PGG are used together, either as a cocktail or sequentially as indicated above, there appeared to be a synergistic effect between the two reagents, resulting in very little degradation of the tissue. It should be noted that the digestion model used in this experiment is a very accelerated and aggressive digestion model.

### Example 5

### Mechanical Testing to Determine the Stiffness of the Treated Tissue

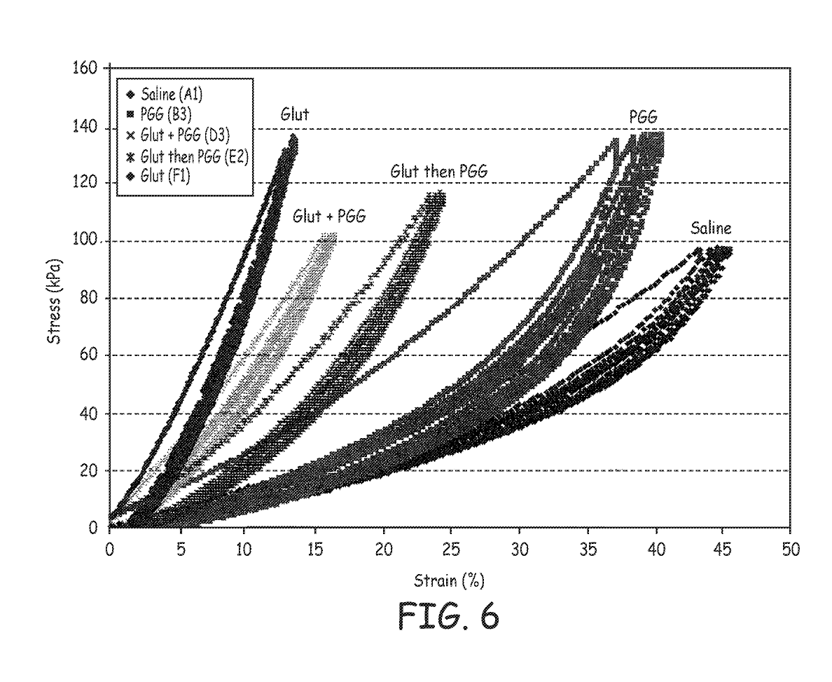

Porcine carotid arteries were treated using the conditions specified in Example 1. The treated tissues were then subjected to uni-axial tensile testing and the results are shown in FIG. 6. The degree of tissue stiffness is indicated by the slope of the curves. The more vertical curve corresponds to more stiffness. The more horizontal curve corresponds to less stiffness. As shown in FIG. 6, the saline treated control tissue is least stiff since it is essentially fresh native tissue. Glut treatment yielded the stiffest tissue. The inclusion of PGG in the treatment process made the tissue slightly less stiff. The results suggest that the stiffness of the treated tissue can be tuned by using different ratio concentration combination of Glut and PGG.

### Example 6

### Mechanical Tests of Treated Aorta Tissue

Porcine suprarenal thoracic aorta was collected from a USDA approved slaughterhouse, transported on ice, and rinsed thoroughly in saline. The rinsed aorta was cut into circumferential rings having a diameter of about 4 cm with about 1.5 cm in height. PGG was dissolved in a 50 mM Na₂HPO₄, pH 6.2 saline buffer at 60° C. to create a 0.06% PGG solution. Similarly, Glut was diluted in the same 50 mM Na₂HPO₄, pH 6.2 saline buffer to create Glut solutions with high Glut (0.20%) and low Glut (0.02%) concentrations. In the case of PGG+Glut groups, the 0.06% PGG solution was first created and allowed to cool to 37° C. before adding Glut to create the final PGG+low Glut (0.06% PGG and 0.02% Glut) and PGG+high Glut (0.06% PGG and 0.20% Glut) solutions. The 50 mM Na₂HPO₄, pH 6.2 saline buffer was used as control. Each sample solution created has 60 mL, which is divided into four portions of 15 mL each to be used to treat aorta samples.

The aorta rings were immersed in different fixing solutions (four duplicating samples for each solution) for 25 min at 37° C. After the treatment, the aorta rings were rinsed in saline. The rinsed treated aorta rings are cut open to create samples for tensile tests that strained the samples in circumferential direction with testing (tensile) speeds equal to about 0.5 mm/sec, using a 10 lb load cell on a Chatillon LTCM-6 testing apparatus. The testing was performed in the same day as collection and treatment of the porcine suprarenal thoracic aorta.

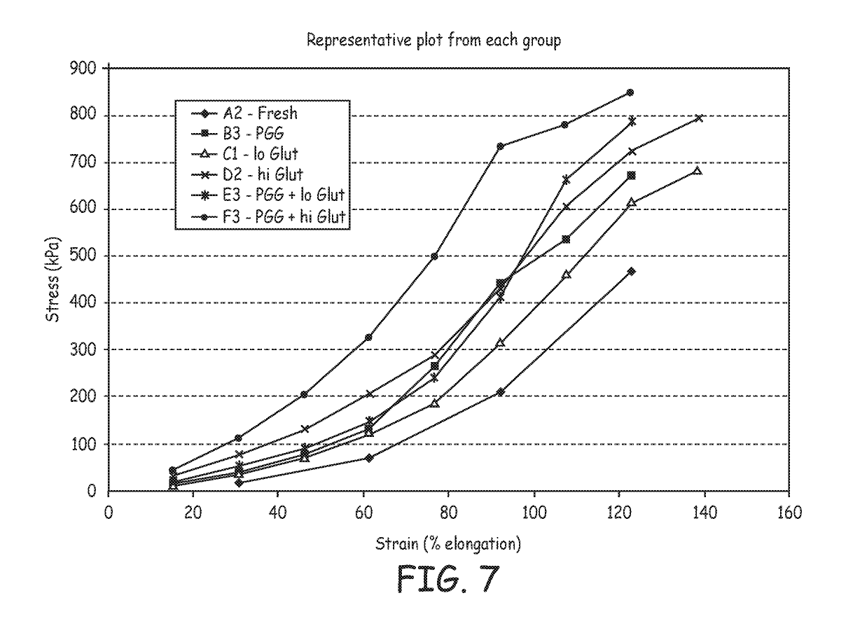

The data collected was a measurement of force vs. displacement which is converted to stress (kPa) vs. strain (% elongation) and plotted in FIG. 7. The control sample (labeled as fresh in FIG. 7) showed the most elongation with increasing stress. The sample treated with 0.02% Glut (labeled as lo Glut) showed less elongation than the control sample, indicating more resistance to tensile strain. The sample treated with 0.06% PGG (labeled as PGG) showed less elongation than the 0.02% Glut treated sample, and is similar to the sample treated with 0.20% Glut (labeled as hi Glut) and the sample treated with 0.06% PGG+0.02% Glut (labeled as PGG+lo Glut) before the tensile strain reached 500 kPa. After the tensile strain reached 500 kPa, the sequence of the degree of elongation among the samples was PGG+lo Glut>hi Glut>PGG+lo Glut. The sample treated with 0.06% PGG and 0.20% Glut (labeled as PGG+hi Glut) showed the least elongation, indicating the most resistance to tensile strain.

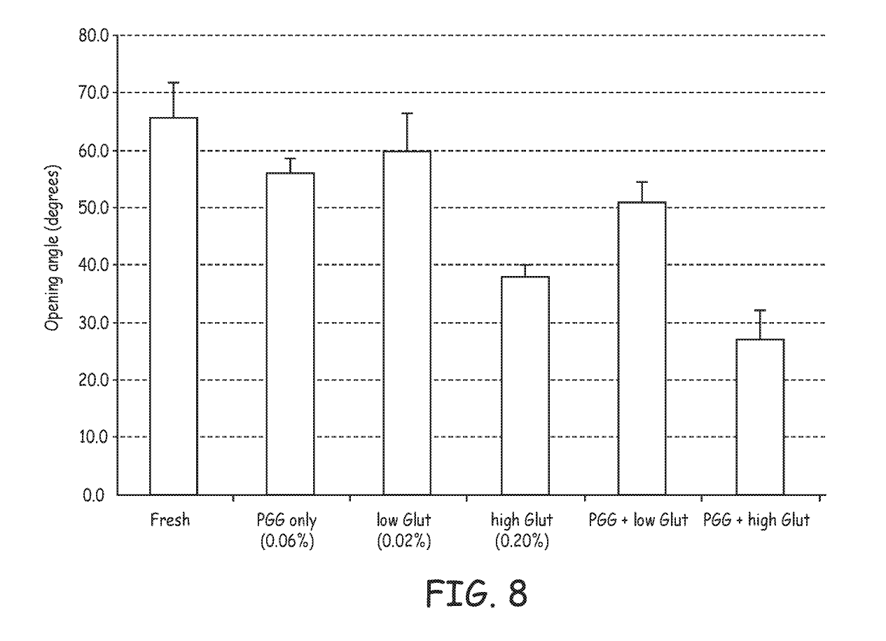

Prior to tensile testing, the treated aorta rings were photographed to obtain ring opening angle, using the method outlined in Example 1. The opening angle of the aorta rings are plotted in FIG. 8. The sequence of the opening angles among the samples was Fresh (control)>lo Glut>PGG>PGG+lo Glut>hi Glut>PGG+hi Glut. The opening angle results are consistent with the results above the 500 kPa strain value from the tensile strain test.

We claim:

1. A method for the stabilization of a blood vessel in a living subject, the method comprising applying an effective amount of a collagen stabilization agent to an isolated section within the interior of the blood vessel, wherein the collagen stabilization agent comprises a compound that is a polyamine, a photo-catalytic dye, genipin, an epoxide, an azide ester, or a combination thereof.

2. The method of claim 1 further comprising aspirating the isolated section of the blood vessel prior to the application of the stabilization agent.

3. The method of claim 1 wherein the stabilization agent is applied for about 1 min to about 4 hours.

4. The method of claim 1 further comprising aspirating the vessel to remove the stabilization agent and applying a second quantity of the stabilization agent after performing the aspiration.

5. The method of claim 4 wherein the stabilization agent applied in the second application has a different composition from the collagen stabilization agent of the first application.

6. The method of claim 1 wherein the collagen stabilization agent is applied simultaneously with an elastin stabilization agent, which comprises a phenolic compound with a hydrophobic core.

7. The method of claim 1 wherein a collagen stabilization agent is applied sequentially with the elastin stabilization agent, which comprises a phenolic compound with a hydrophobic core.

8. The method of claim 1 further comprising an aspiration, a rinse, or combinations of the aspiration and rinse steps.

9. The method of claim 8 wherein the rinse comprises an amino acid, serum albumin or a combination thereof.

10. The method of claim 1 wherein the isolated section of the blood vessel comprises aneurysm.

11. A method for in vivo stabilization of a blood vessel, the method comprising:
rinsing an isolated section of the blood vessel with a toxicity reducing composition following the treatment of the isolated section of blood vessel with a multifunctional aldehyde,
wherein the toxicity reducing composition comprises a toxicity reducing agent.

12. The method of claim 11 wherein the multifunctional aldehyde is glutaraldehyde.

13. The method of claim 11 wherein the toxicity reducing agent comprises a nonionic surfactant.

14. The method of claim 11 wherein the toxicity reducing composition comprises amines.

15. The method of claim 14 wherein the amine comprises an amino acid.

16. The method of claim 11 wherein the toxicity reducing composition comprises inorganic sulfur-oxygen containing anions, organic sulfates, ammonium salts, surfactants, or a combination thereof.

17. The method of claim 16 wherein the sulfur-oxygen anions comprises sulfate anions SO₄⁻², thiosulfate anions S₂O₄⁻², bisulfate HSO₄⁻, or a combination thereof, wherein the organic sulfates comprises methyl sulfate CH₃O₄S⁻, dimethyl sulfate (CH₃)₂O₄S and dodecyl sulfate CH₃(CH₂)₁₁O₄S⁻ as well as protonated forms thereof, wherein the ammonium salt comprises ammonium chloride, ammonium hydroxide or other suitable salt thereof, and wherein the surfactants comprises aliphatic fatty acid esters, polypropyleneglycol fatty acid esters, glycerol fatty acid esters, polyalkylene ethers, polyoxyethylene oleyl ether, polyoxyethylene cetyl ether, polyethylene glycol p-isooctyl phenyl ethers, polyoxyethylene sorbitan esters, or a combination thereof.

18. The method of claim 11 wherein the toxicity reducing agents have a concentration from about 0.005 molar (M) to about 3 M.

19. A method for the stabilization of a blood vessel in a living subject, the method comprising applying an effective amount of a collagen stabilization agent and an elastin stabilization agent to an isolated section within the interior of the blood vessel,
wherein the blood vessel comprises collagen having a plurality of reactive groups and elastin,
wherein the collagen stabilization agent comprises a delivery vehicle and a compound that reacts with reactive groups of the collagen, and
wherein the elastin stabilization agent comprises a delivery vehicle and a compound that crosslinks elastin.

20. The method of claim 19 wherein the compound in the collagen stabilization agent is an aldehyde with at least two aldehyde groups, a polyamine with carbodiimide, a photo-catalytic dye, genipin, an epoxide, an azide ester, or a combination thereof.
